(19) United States
(12) Patent Application Publication
(10) Pub. No.: US 2022/0084640 A1
(43) Pub. Date: Mar. 17, 2022

(54) CUSTOM DATA FILES FOR PERSONALIZED MEDICINE

(71) Applicant: Illumina Software, Inc., San Diego, CA (US)

(72) Inventors: Egan Jackson Lohman, Bozeman, MT (US); Christopher Karl Edlund, Carlsbad, CA (US); Dwight Thomas Baker, San Diego, CA (US); Jeremy Joseph Ward, Escondido, CA (US)

(21) Appl. No.: 17/447,554

(22) Filed: Sep. 13, 2021

Related U.S. Application Data
(60) Provisional application No. 63/078,215, filed on Sep. 14, 2020.

(51) Int. Cl. G16H 10/60; G16H 70/40; G16B 20/00; H04L 9/32; G06F 11/10

(52) U.S. Cl. CPC G16H 10/60; G16H 70/40; G06F 11/1004; H04L 9/3236; G16B 20/00

(57) ABSTRACT

Methods and systems are disclosed which can gather large data sets from nucleic acid sequencing technologies and devices, filter relevant genomic information and sequence variant information of biological samples from files of various formats, generate a custom data file having only relevant information in a standardized format, and provide the generated information to downstream analysis for personalized medicine use.

### CROSS-REFERENCE TO RELATED APPLICATIONS

This application claims priority to U.S. Provisional Application No. 63/078,215, filed Sep. 14, 2020, the content of which is incorporated by reference in its entirety.

### REFERENCE TO COMPUTER PROGRAM LISTING

This application is submitted with a computer program listing appendix including one file entitled “biomarker_definitions.schema.txt” created Jul. 19, 2019, (2,139 bytes), one file entitled “nirvana_definitions.schema.txt” created Aug. 5, 2019, (6,721 bytes), one file entitled “sample_analysis_results.txt” created Aug. 12, 2019, (16,154 bytes), one file entitled “sample_analysis_results.schema.txt” created Jul. 24, 2019, (9,368 bytes), and one file entitled “variant_definitions.schema.txt” created Aug. 12, 2019, (6,857 bytes), and is incorporated by reference herein for all purposes.

### BACKGROUND OF THE INVENTION

### Field of the Invention

Aspects of the invention relate to methods and systems for generating a custom data file. In particular, embodiments include methods and systems for gathering, analyzing, filtering, aggregating, and storing genomic information and sequence variant information of biological samples from a plurality of files having various formats into a single standard file.

### Description of the Related Art

Technology for determining the sequence of an organism's DNA sequence and RNA expression has progressed dramatically. With the development of dye-terminator based sequencing (Sanger sequencing) and related automated technologies, the field of nucleic acid sequencing took a giant step forward. The advent of dye-based technologies and instrumentation and automated sequencing methods required development of related software and data processes to manage all the generated data.

Genetic sequencing has become an increasingly important area of genetic research, promising future uses in diagnostic and other applications. In general, genetic sequencing involves determining the order of nucleotides for a nucleic acid such as a fragment of RNA or DNA. Relatively short sequences are typically analyzed, and the resulting sequence information may be used in various bioinformatics methods to logically fit fragments together to reliably determine the sequence of much more extensive lengths of genetic material from which the fragments were derived. Automated, computer-based examinations of characteristic fragments have been developed and have been used more recently in genome mapping, identification of genes and their function, and so forth.

In recent years, the cost of sequencing and the time required to determine the sequence of a genetic sample has dramatically decreased. Samples that previously required months to sequence can now be sequenced in a matter of days or weeks. Whole genome sequencing or partial genome sequencing can now be performed at a much lower cost, which removes the cost barrier for many consumers.

Besides the data gathered during and after sequencing, the genomic analysis workflow from sample extraction to reporting of the data analysis may involve the generation of a significant amount of information and various manifests for tracking sample and content information. In addition, different sequencing assays generate different data outputs, but having multiple different data outputs can be clunky and duplicative. Thus, there is a need for improved techniques in the management of such information before, during, and after the genomic analysis workflow.

### SUMMARY OF THE INVENTION

The systems, devices, kits, and methods disclosed herein each have several aspects, no single one of which is solely responsible for their desirable attributes. Without limiting the scope of the claims, some prominent features will now be discussed briefly. Numerous other embodiments are also contemplated, including embodiments that have fewer, additional, and/or different components, steps, features, objects, benefits, and advantages. The components, aspects, and steps may also be arranged and ordered differently. After considering this discussion, and particularly after reading the section entitled “Detailed Description,” one will understand how the features of the devices and methods disclosed herein provide advantages over other known devices and methods.

In one aspect, the disclosed technology relates to a computer-implemented method of generating a custom file. The method comprises receiving a query for information associated with a desired sample. The method further comprises determining a schema for structuring the custom file. The method further comprises obtaining, according to the schema, a plurality of nucleic acid sequencing analysis files, wherein each one of the plurality of nucleic acid sequencing analysis files comprises nucleic acid sequence information, genetic variant information, gene expression information, or any combination thereof, of a plurality of biological samples, wherein the plurality of biological samples comprise the desired sample. The method further comprises, for each one of the plurality of nucleic acid sequencing analysis files: determining, according to the schema, a plurality of data objects in the nucleic acid sequencing analysis file to be stored in the custom file; determining, according to the schema, a plurality of custom data fields in the custom file to store the data objects; and storing the data objects in the custom data fields. The method further comprises generating a checksum by evaluating a cryptographic hash function for a portion of the custom file according to the schema. The method further comprises storing the checksum in the custom file.

In some embodiments, determining a schema for structuring the custom file comprises: choosing a schema from a plurality of pre-defined schemas; optionally, receiving user modifications for modifying the schema; and storing the user modifications and a version value associated with the schema in the custom file.

In some embodiments, obtaining, according to the schema, a plurality of nucleic acid sequencing analysis files comprises: searching a database for a plurality of files comprising one or more keywords specified by the schema; and copying the plurality of files.

In some embodiments, determining, according to the schema, a plurality of data objects in the nucleic acid sequencing analysis file to be stored in the custom file comprises: parsing the nucleic acid sequencing analysis file; identifying, according to the schema, the plurality of data objects to be stored; and extracting the plurality of data objects.

In some embodiments, each of the nucleic acid sequencing analysis files further comprises at least one of: sequencing device condition, sequencing related data, analysis software information, analysis pipeline information, base calls, run quality control metrics, DNA quality control metrics, RNA quality control metrics, DNA small variants outputs, copy number variant outputs, RNA fusion outputs, DNA fusion outputs, splice variant outputs, tumor mutational burden biomarker outputs, and microsatellite instability biomarker outputs. In some embodiments, the sequencing device condition comprises sequencing parameters and/or information about errors in the sequencing device.

In some embodiments, each of the nucleic acid sequencing analysis files further comprises at least one of: sample preparation related data, sample identification number, sample manifest, patient identify, tissue type, genomic area of interest, disease information, and treatment information.

In some embodiments, the method further comprises: receiving a user input associated with the desired sample; determining, according to the schema, a plurality of data objects in the user input to be stored in the custom file; determining, according to the schema, a plurality of custom data fields in the custom file to store the data objects; and storing the data objects in the custom data fields. In some embodiments, the user input associated with the desired sample comprises at least one of: sample preparation related data, sample identification number, sample manifest, patient identify, tissue type, genomic area of interest, disease information, and treatment information.

In some embodiments, the cryptographic hash function is a MD5 hash function, a MD6 hash function, a SHA-1 hash function, a SHA-256 hash function, or a SHA-512 hash function.

In some embodiments, the method further comprises: generating a verification value by adding or multiplying the checksum by a number; and storing the verification value in the custom file. In some embodiments, the number is π.

In some embodiments, the portion of the custom file according to the schema comprises a plurality of custom data fields declared by the schema as not permitting user corrections. In some embodiments, the method may further comprise: generating an additional checksum by evaluating a cryptographic hash function for an additional portion of the custom file according to the schema, wherein the additional portion of the custom file comprises a plurality of custom data fields declared by the schema as permitting user corrections; and storing the additional checksum in the custom file.

In some embodiments, the method further comprises: receiving and storing a plurality of user changes to a plurality of custom data fields; updating the checksum by re-evaluating the cryptographic hash function for the portion of the custom file according to the schema; and storing the updated checksum in the custom file.

In some embodiments, some of the nucleic acid sequencing analysis files are compressed.

In some embodiments, the method further comprises: compressing and/or encrypting the custom file.

In some embodiments, the custom file is in text-based JavaScript Object Notation (JSON) format or binary JSON format.

In some embodiments, each of the nucleic acid sequencing analysis files is in one of JSON, CSV, TSV, XML, NirvanaJSON, VCF, CSVVCF, or SpliceJSON format.

In some embodiments, the method is implemented in a cloud computing environment.

In another aspect, the disclosed technology relates to a database comprising a plurality of files, wherein each of the plurality of files is generated according to the disclosed method.

In yet another aspect, the disclosed technology relates to a system for generating a custom file, comprising: a memory storing instructions to implement the disclosed method; and one or more processors configured to execute the instructions.

In yet another aspect, the disclosed technology relates to a computer program product for generating a custom file, comprising a computer readable storage medium having program instructions to implement the disclosed method.

### BRIEF DESCRIPTION OF THE DRAWINGS

FIG. 1 illustrates an exemplary system for generating a SARJ file from sequencing and variant analyses results for downstream genomic analyses.

FIG. 2A shows an exemplary portion of the SARJ schema. FIG. 2B shows an exemplary portion of a SARJ file.

FIG. 3 illustrates an exemplary workflow of one method of generating a SARJ file.

### DETAILED DESCRIPTION

All patents, applications, published applications and other publications referred to herein are incorporated herein by reference to the referenced material and in their entireties. If a term or phrase is used herein in a way that is contrary to or otherwise inconsistent with a definition set forth in the patents, applications, published applications and other publications that are herein incorporated by reference, the use herein prevails over the definition that is incorporated herein by reference.

Embodiments relate to methods and systems for generating a custom file by gathering, analyzing, filtering, aggregating, and storing genomic information and sequence variant information of biological samples from a plurality of files having various formats. Disclosed methods and processes may be applicable to the fields of genomic DNA and RNA sequencing, whole genome sequencing, whole genome haplotyping, cancer sequencing, resequencing, gene expression analysis, drug discovery, disease discovery and diagnosis, targeted resequencing, therapeutics and disease related treatment response, prognostics, disease correlations, evolutionary genetics, etc. Disclosed methods may further be applicable to other fields, such as signal processing or and information retrieval and data compression fields, such as when experiments or data acquisition processes produce large datasets and a variety of analysis results and file formats.

Embodiments of the invention relate to systems and methods for inputting a variety of different files containing genetic information and outputting a standard file, termed herein a Sample Analysis Results JSON (SARJ) file, that can be used for a variety of genomic analyses. For example, in one embodiment, genetic sequence information is received from DNA sequencing of a particular biological sample. That genetic sequence information is analyzed to determine variants or other features of the genetic sequence information. The data output of that variant analysis may be in the form of a variety of different file formats, including DNA variant files, RNA variant files, quality control metrics, biomarkers, and other sample information such as the date/time/place where the sample was taken. The data output from the variant files may then be input into a system to generate the SARJ file using one or more electronic schema defining the structure of the data output being stored as a SARJ file. In one embodiment, once the SARJ file is generated by a SARJ generator system, the system calculates a checksum that is appended to the SARJ file to prevent the file from being altered. For example, the data within the SARJ file may be run through a cryptographic hash function to generate the checksum and that checksum stored in the header of the SARJ file.

Using the standard SARJ file can improve the efficiency of downstream genomic analyses. Currently, different variant analysis tools and software programs from different providers may store their data output in a variety of different file formats, such as bam, bcl, vcf, csv, xml, JSON, or SpliceJSON. These data output files may not contain the same kinds of information, or may contain information that are not needed for downstream genomic analyses. For example, one data output file may contain RNA variant information of a few different tissue types of one patient, and another data output file may contain DNA variant information of that patient together with a few other people. Furthermore, these data output files may be compressed or encrypted. The SARJ generator can automatically search for relevant variant analysis data output files and extract only the desired information, as defined by the electronic schema. The resulting SARJ file presented to the downstream analyses will be in a standard format and will contain only the desired information, for example, information of a particular tissue type of only one patient. Therefore, the downstream genomic analyses do not have to work with different file formats, locate the relevant files, or parse through the files to find the desired information. For example, the downstream genomic analysis can quickly identify a disease related to the particular tissue type of that patient and select treatments for the disease, based on biomarkers reported in the SARJ file.

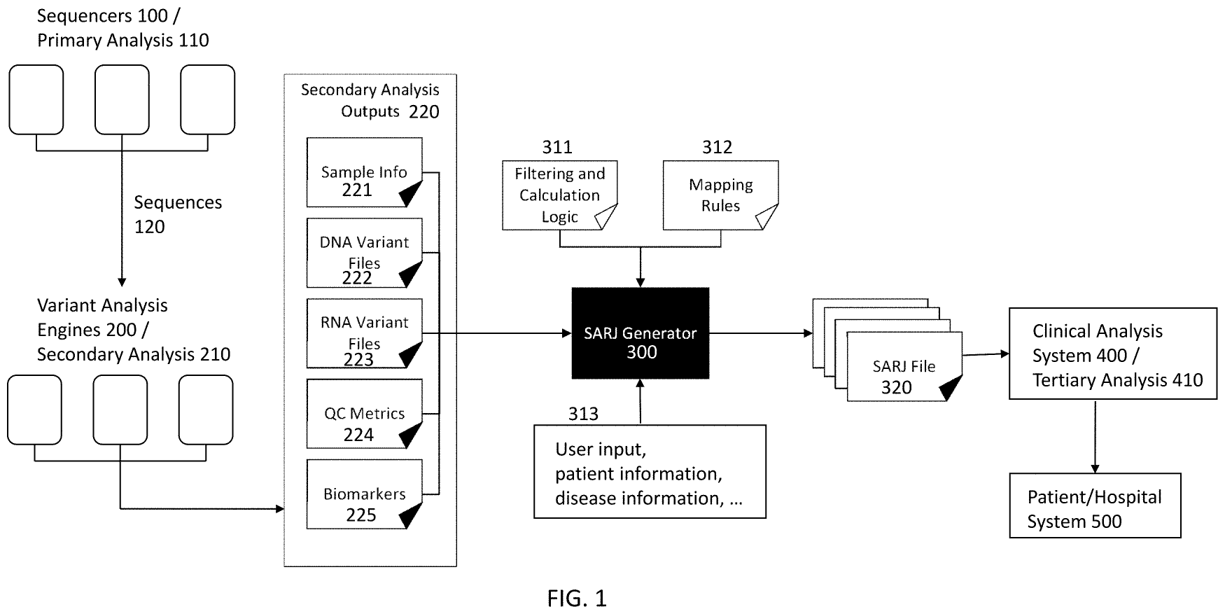

One embodiment is shown in the flow diagram of FIG. 1. As shown, FIG. 1 illustrates an exemplary workflow of generating a standardized SARJ file 320 for personalized medicine from a plurality of nucleic acid sequencing analysis output files 220.

The exemplary workflow starts from adding biological samples to assay instruments, for example, nucleic acid sequencers 100. In some embodiments, one of the assay instruments may be a microarray instrument, a scanner, or a fluorescent imaging instrument. Data generated by the assay instruments may be computationally analyzed either directly on the assay instruments (e.g., via software stored on or loaded onto the sequencers 100) or indirectly (e.g., on a computer system or storage device, a desktop computer, a laptop computer, or a server that is operationally connected to an assay instrument). In some embodiments, the sequencers 100 include separate sample processing devices and associated computers. In alternative embodiments, these may be implemented as a single device. In some embodiments, the associated computers may be local to or networked with the sample processing devices. In other embodiments, the associated computers may be capable of communicating with the sequencers 100 through a cloud computing environment.

In some embodiments, the biological samples are tumor samples from a patient. The tumor samples may be prepared for next-generation sequencing (NGS) using Illumina's TruSight Oncology 500 assay before being added to the assay instruments. In some embodiments, both DNA sequencing and RNA sequencing (RNASeq) may be performed to determine the gene structure and the transcriptome data of the biological samples.

The sequencers 100 may perform primary analysis 110 to determine the nucleic acid sequences 120 in the biological samples. In some embodiments, the output sequences 120 may comprise a large number of short sequences, called “reads”, plus metadata associated with each read and a quality score that estimates the confidence of each nucleotide base in a read.

The primary analysis stage processing 110 functions to translate physical signals detected inside the sequencer into “reads” of nucleotide sequences with associated quality or confidence scores, e.g. FASTQ format files, or other formats containing sequence and usually quality information. Primary analysis may be specific to the sequencing technology employed. In various sequencers, nucleotides are detected by sensing electrical charges, electrical currents, or radiated light. In some embodiments, primary analysis may include: signal processing to amplify, filter, separate, and measure sensor output; data reduction, such as by quantization, decimation, averaging, transformation, etc.; image processing or numerical processing to identify and enhance meaningful signals, and associate them with specific reads and nucleotides (e.g. image offset calculation, cluster identification); data correction and optimization methods to compensate for sequencing technology artifacts (e.g. phasing estimates, cross-talk matrices); Bayesian probability calculations; hidden Markov models; base calling (selecting the most likely nucleotide at each position in the sequence); base call quality (confidence) estimation, and the like.

Once the sequences 120 are produced by the sequencers 100, the sequences 120 are transmitted to variant analysis engines 200. The variant analysis engines 200 perform a secondary analysis 210, and produce secondary analysis output files 220.

Secondary analysis 210 determines the content of the sequenced sample DNA or RNA, such as by mapping and aligning reads to a reference genome, sorting, duplicate marking, base quality score recalibration, local re-alignment, and variant calling. Performing a secondary analysis on a subject's sequenced DNA may, for example, determine how the subject's DNA varies from that of the reference.

In some embodiments, secondary analysis 210 may involve de novo sequence assembly, comparison of test genome sequences to those of reference genomic sequences, determining the presence or absence of single-nucleotide variants (SNVs), insertions, deletions, single-nucleotide polymorphism (SNPs) and other genomic variant mutations in a genome, comparing test RNA sequences to those of reference RNA sequences, determining splice variants, RNA sequence anomalies, presence or absence of RNA sequences, or resequencing of a genome.

In some embodiments, the variant analysis engines 200 may be any general-purpose computers implementing analysis software for analyzing sequencing datasets, for example software programs such as Pipeline, CASAVA and GenomeStudio data analysis software (IIlumina®, Inc.), SOLID™, DNASTAR® SeqMan® NGen® and Partek® Genomics Suite™ data analysis software (Life Technologies), Feature Extraction and Agilent Genomics Workbench data analysis software (Agilent Technologies), Genotyping Console™, Chromosome Analysis Suite data analysis software (Affymetrix®). In alternative embodiments, a single device may perform both the primary analysis and the secondary analysis. The secondary analysis outputs 220 generated from various software programs may take the form of FASTQ files, binary alignment files (bam) *.bcl, *.vcf, and/or *.csv files. The secondary analysis outputs 220 may be of JSON, CSV, TSV, XML, NirvanaJSON, VCF, CSVVCF, or SpliceJSON format. In some embodiments, the secondary analysis output files 220 may be compressed.

In some embodiments, secondary analysis output files 220 may comprise at least one of: sequencing device condition, sequencing related data, analysis software information, analysis pipeline information, base calls, run quality control metrics, DNA quality control metrics, RNA quality control metrics, DNA small variants outputs, copy number variant outputs, RNA fusion outputs, DNA fusion outputs, splice variant outputs, tumor mutational burden biomarker outputs, and microsatellite instability biomarker outputs. The sequencing device condition may comprise sequencing parameters and/or information about errors in the sequencing device. In some embodiments, secondary analysis output files 220 may include one or more of the following: run quality control (QC) metrics, DNA QC metrics, RNA QC metrics, DNA small variants outputs, copy number variant outputs, RNA fusion outputs, DNA fusion outputs, splice variant outputs, additional variants, tumor mutational burden biomarker outputs, microsatellite instability biomarker outputs or additional biomarkers, and at least one of: sample preparation related data, sample identification number, sample manifest, patient identify, tissue type, genomic area of interest, disease information, and treatment information.

Once the secondary analysis output files 220 are available, a SARJ generator (SARJeant) 300 may gather and analyze a plurality of sequencing analysis output files 220. The SARJ generator 300 can filter, extract and aggregate relevant data from these files, and generate a single Sample Analysis Results JSON (SARJ) file 320 for each desired biological sample.

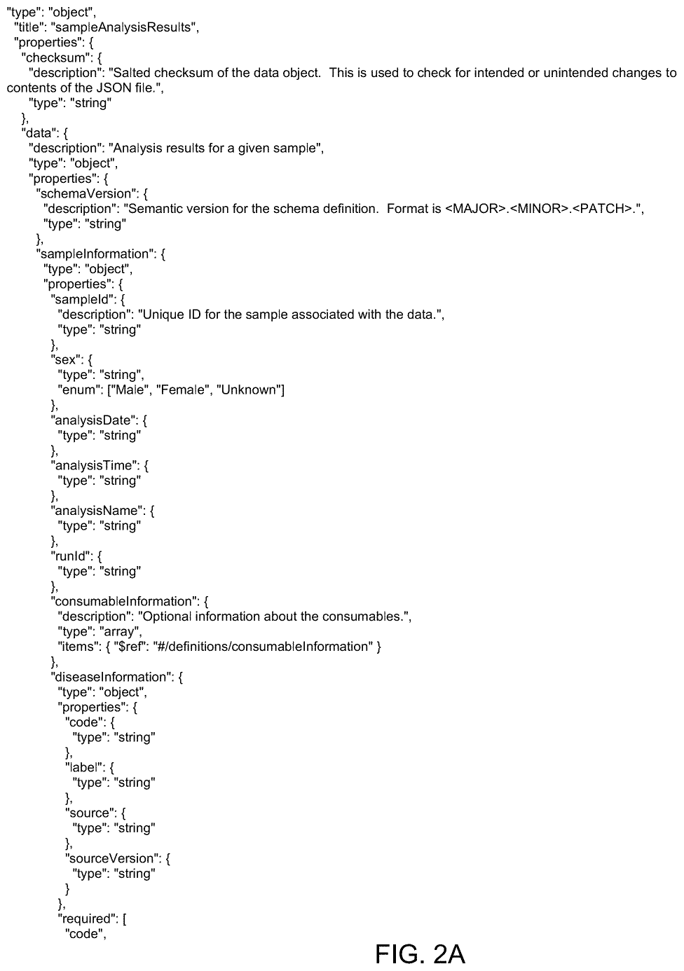

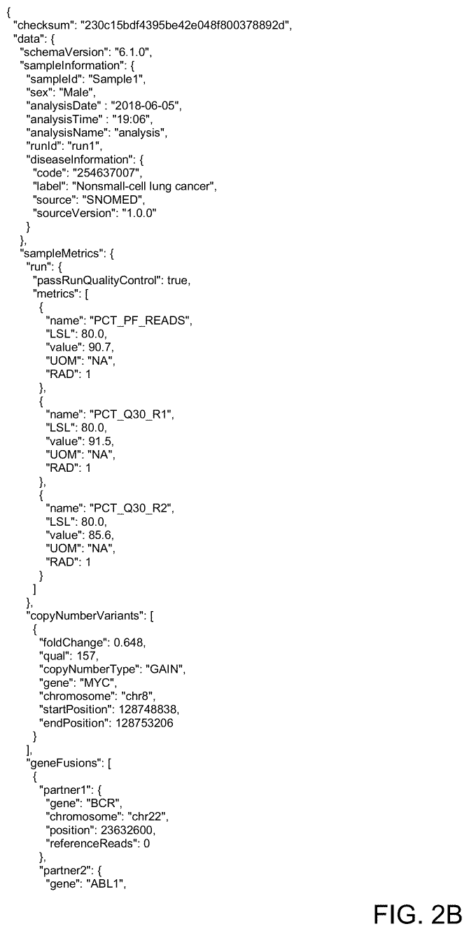

In some embodiments, the SARJ generator 300 may receive a query for information associated with a desired biological sample, and determine a schema for structuring the SARJ file 320. The schema may be chosen from a plurality of pre-defined schemas, and can allow user modifications. One example of a schema is shown in FIG. 2A. The user modifications and a version value associated with the schema will be stored in the SARJ file 320.

The SARJ generator 300 may obtain a plurality of secondary analysis output files 220 that are associated with the desired biological sample, for example a sample information file 221, several DNA variant files 222, several RNA variant files 223, files that contain quality control (QC) metrics 224 and files that contain biomarkers 225. The secondary analysis output files 220 may additionally contain data associated with other biological samples. In some embodiments, to obtain the secondary analysis output files 220, the SARJ generator 300 may search a database for a plurality of files comprising one or more keywords specified by the schema, and copying the plurality of files.

The SARJ generator 300 may then determine the data objects in the secondary analysis output files 220 to be stored in the SARJ file 320, according to the filtering and calculation logic 311. In some embodiments, to determine the data objects, the SARJ generator 300 may parse and analyze the secondary analysis output files 220, and extract the data objects identified according to the logic 311. In some embodiments, the SARJ generator 300 may receive a user input associated with the desired sample which includes a plurality of data objects to be stored.

The SARJ generator 300 may also determine the custom data fields used to store the data objects in the SARJ file 320, according to the mapping rules 312. The SARJ generator 300 may then store the data objects in the custom data fields. In some embodiments, the SARJ generator 300 may store a plurality of data objects from a user input.

The filtering and calculation logic 311 and the mapping rules 312 may be customizable.

In some embodiments, the user input associated with the desired sample may comprise at least one of: sample preparation related data, sample identification number, sample manifest, patient identify, tissue type, genomic area of interest, disease information, and treatment information.

To authenticate or validate the SARJ file 320 after transmission, the SARJ generator 300 may generate a checksum by evaluating a cryptographic hash function for a portion of the SARJ file 320, and store the checksum in the SARJ file 320. In some embodiments, the checksum is salted by adding or multiplying the checksum by a number. The number may be π. In some embodiments, the cryptographic hash function is a MD5 hash function, a MD6 hash function, a SHA-1 hash function, a SHA-256 hash function, or a SHA-512 hash function. In some embodiments, the SARJ generator 300 may checksum a portion of the SARJ file 320 which is a section declared by the schema as not permitting user corrections. In some embodiments, the SARJ generator 300 may generate an additional checksum by evaluating a cryptographic hash function for an additional portion of the SARJ file 320, which comprises a plurality of custom data fields declared by the schema as permitting user corrections. In some embodiments, the SARJ generator 300 may receive and store a plurality of user changes to a plurality of custom data fields, and allow users to update the checksum by re-evaluating the cryptographic hash function and store the updated checksum in the custom file.

In some embodiments, the SARJ file 320 may be in text-based JavaScript Object Notation (JSON) format or binary JSON format. In some embodiments, the SARJ generator 300 may compress and/or encrypt the SARJ file 320 before sending the file to downstream processing.

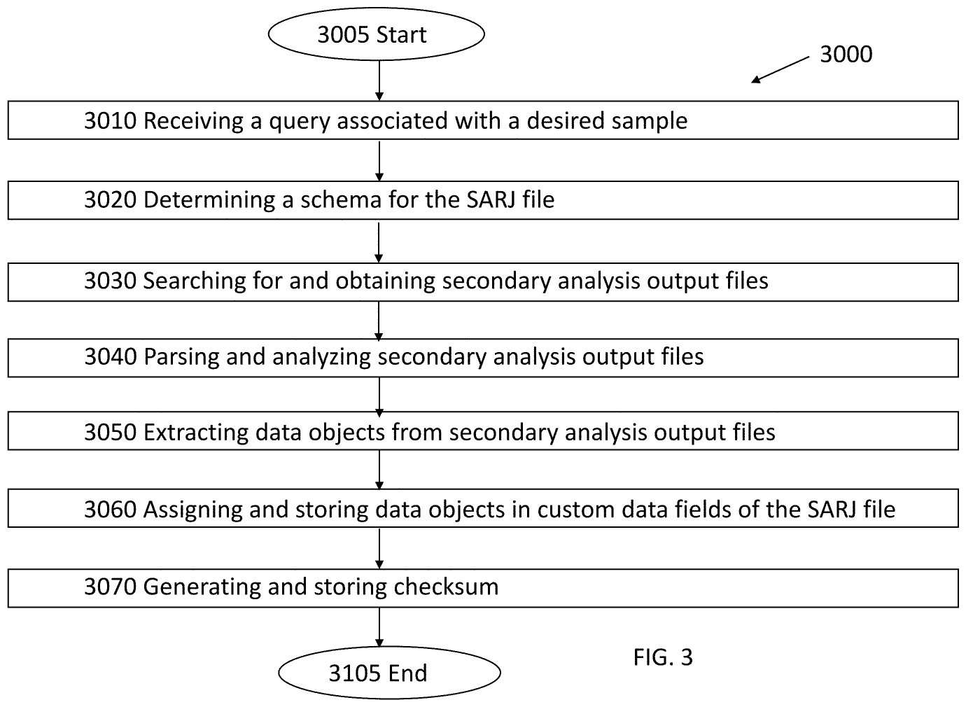

In one embodiment, the SARJ generator 300 creates the SARJ file 320 according to the exemplary workflow 3000 of one method illustrated in FIG. 3. As shown, the process 3000 begins at a start state 3005 and then moves to a state 3010, where a query for information associated with a desired sample is received. The process then moves to a state 3020 that determines an electronic schema for structuring a custom SARJ file to be created for the desired sample. Determining an electronic schema may involve choosing a schema from a plurality of pre-defined schemas and/or receiving user modifications for modifying the schema. In some embodiments, the schema is created offline to match the requirements of the desired SARJ file 320 outputs. In alternative embodiments, the schema is selected dynamically or online. The user modifications and a version value associated with the schema may be stored in the SARJ file. After the electronic schema is determined, the process then moves to a state 3030 where a plurality of nucleic acid sequencing analysis or secondary analysis output files are obtained according to the schema. Obtaining the secondary analysis output files may involve searching a database for one or more keywords specified by the schema. After the secondary analysis output files are obtained, the process then moves to a state 3040 where the secondary analysis output files are analyzed. The secondary analysis output files are parsed, and a plurality of desired data objects or relevant information to be stored are identified according to the schema. The process then moves to a state 3050 that extracts and/or copies the plurality of desired data objects or relevant information from the secondary analysis output files. The process further moves to a state 3060 that determines the custom data fields in the SARJ file corresponding to the desired data objects, and stores the desired data objects in the corresponding custom data fields. After the custom data fields of the SARJ file have been assigned, the process then moves to a state 3070, where a checksum is generated for a portion of the custom SARJ file, and the checksum is stored in the SARJ file. For example, the schema may declare that some of the custom data fields of the SARJ file does not permit user corrections, such that a cryptographic hash function will be evaluated on this portion of the SARJ file to generate a checksum. The process 3000 then terminates at an end state 3105. One example of a SARJ file 320 is shown in FIG. 2B.

Once the SARJ file 320 is generated, the SARJ generator 300 may send it to a downstream clinical analysis system 400 for performing tertiary analysis 410 (e.g. tumor profiling) and further reporting.

In some embodiments, the SARJ file 320 may be accessed by the clinical analysis system 400 through security parameters such as a password-protected client account in a cloud computing environment or the association with a particular institution or IP address. The SARJ file 320 may be accessed by the clinical analysis system 400 by downloading one or more files from the cloud computing environment or by logging into a web-based interface or software program that provides a graphical user display in which the SARJ file 320 is depicted as text, images, and/or hyperlinks. In some embodiments, the SARJ file 320 may be provided to users in the form of data packets transmitted via a communications link or network.

In some embodiments, the clinical analysis system 400 may be designed to deliver in-vitro diagnostic (IVD) solutions to improve the management of cancer patients in the clinic. In some embodiments, the clinical analysis system 400 may develop cancer companion diagnostics (CDx) useful for therapeutics or companion therapeutics. In some embodiments, the clinical analysis system 400 may identify biomarkers for targeted therapies for cancer patients, perform treatment selection through response monitoring which allows physicians to follow the evolution of a patient's tumor over time through the downstream patient/hospital system 500. In some embodiments, the clinical analysis system 400 may analyze the biology that drives cancer predisposition and proliferation that supports the development of targeted therapeutics and multi-analyte tumor analysis. In some embodiments, the clinical analysis system 400 may be used for discovery of novel methods to monitor cancer treatment and recurrence and developing precision medicine or personalized medicine.

In some embodiments, the tertiary analysis 410 extracts medical or research implications from the nucleic acid sequence and variant information in the SARJ file 320. In some embodiments, the tertiary analysis 410 may include genome-wide variation analysis, gene function analysis, protein function analysis, e.g., protein binding analysis, quantitative and/or assembly analysis of genomes and/or transcriptomes, as well as various diagnostic, and/or prophylactic and/or therapeutic evaluation analyses.

In some embodiments, the tertiary analysis 410 may predict the potential for the occurrence of a diseased state due to a genetic abnormality. In some embodiments, the tertiary analysis 410 may identify candidates for clinical trials. In some embodiments, the tertiary analysis 410 may predict the likelihood of success of a prophylactic or therapeutic modality based on how a prophylactic or therapeutic is expected to interact with the patient's genomic or transcriptomic information. In some embodiments, the tertiary analysis 410 may interpret the SARJ file 320, such as for determining what the data means with respect to identifying what diseases a patient may have, and/or for determining what treatments or lifestyle changes a patient may want to employ so as to ameliorate or prevent a diseased state. In some embodiments, a subject's genetic sequence or their variant calls may be analyzed to determine clinically relevant genetic markers that indicate the existence or potential for a diseased state, and/or the efficacy of a proposed therapeutic or prophylactic regimen may have on the subject.

In some embodiments, once the tertiary analysis 410 is performed by the clinical analysis system 400, the result of tertiary analysis 410 is optionally reported to a downstream patient/hospital system 500.

In some embodiments, the patient/hospital system 500 may use the result of tertiary analysis 410 to diagnose a disease or its potential, perform clinical interpretation (e.g., looking for markers that represent a disease variant), or determine whether a subject should be included or excluded in various clinical trials. In some embodiments, the patient/hospital system 500 may query for a certain type of information that are known to be associated with a certain disease by determining if one or more genetic based diseased markers are included in the result of the tertiary analysis 410.

Other aspects and advantages of the disclosure will become apparent from this detailed description taken in conjunction with the accompanying drawings which illustrate, by way of example, the principles of the disclosure.

While only certain features of the invention have been illustrated and described herein, many modifications and changes will occur to those skilled in the art. It is, therefore, to be understood that the appended claims are intended to cover all such modifications and changes as fall within the true spirit of the invention.

Various modification and variation of the described methods and compositions of the invention will be apparent to those skilled in the art without departing from the scope of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the following claims.

Embodiments of the present techniques are described herein by reference to sample preparation data generated by a sample preparation device, sequencing data generated by a sequencing device, and/or information related to generating, analyzing, and reporting this type of data. The disclosure is not, however, limited by the advantages of the aforementioned embodiment. The present techniques may alternatively or additionally be applied to devices capable of generating other types of high throughput biological data, such as microarray data. Microarray data may be in the form of expression data, and the expression data may be stored, processed, and/or accessed by primary or secondary users in conjunction with the cloud computing environment as provided herein. Other devices that can be used include, but are not limited to, those capable of generating biological data pertaining to enzyme activity (e.g. enzyme kinetics), receptor-ligand binding (e.g. antibody binding to epitopes or receptor binding to drug candidates), protein binding interactions (e.g. binding of regulatory components to nucleic acid enzymes), or cell activity (e.g. cell binding or cell activity assays).

Advantages of practicing the methods and systems as described herein can provide investigators with more efficient systems that utilize fewer computer resources while maximizing data analysis time, thereby providing investigators with additional tools for determining the presence or absence of disease related genomic anomalies which may be used by a clinician to diagnose a subject with a disease, to provide a prognosis to a subject, to determine whether a patient is at risk of developing a disease, to monitor or determine the outcome of a therapeutic regimen, and for drug discovery. Further, information gained by practicing computer implemented methods and systems comprising processes as described herein finds utility in personalized healthcare initiatives wherein an individual's genomic sequence may provide a clinician with information unique to a patient for diagnosis and specialized treatment. Therefore, practicing the methods and systems as described herein can help provide investigators with answers to their questions in shorter periods of time using less valuable computer resources.

Sequencing Technologies

In some embodiments, the sequencers 100 are provided by Illumina®, Inc. (NovaSeq 6000, NextSeq 550, NextSeq 1000, NextSeq 2000, HiSeq 1000, HiSeq 2000, Genome Analyzers, MiSeq, HiScan, iScan, BeadExpress systems), Applied Biosystems™ Life Technologies (ABI PRISM® Sequence detection systems, SOLID™ System), Roche 454 Life Sciences (FLX Genome Sequencer, GS Junior), Applied Biosystems™ Life Technologies (ABI PRISM® Sequence detection systems, SOLiD™ System), or Ion Torrent® Life Technologies (Personal Genome Machine sequencer).

The sequencers 100 may be implemented according to any sequencing technique, such as those incorporating sequencing-by-synthesis methods described in U.S. Patent Publication Numbers 2007/0166705, 2006/0188901, 2006/0240439, 2006/0281109, 2005/0100900, U.S. Pat. No. 7,057,026, PCT Publication Numbers WO 2005/065814, WO 2006/064199, and WO 2007/010251, the disclosures of which are incorporated herein by reference in their entireties. Alternatively, sequencing by ligation techniques may be used in the sequencers 100, such as described in U.S. Pat. Nos. 6,969,488, 6,172,218, and 6,306,597, the disclosures of which are incorporated herein by reference in their entireties. Sequencing by ligation techniques use DNA ligase to incorporate oligonucleotides and identify the incorporation of such oligonucleotides. Some embodiments can utilize nanopore sequencing, whereby target nucleic acid strands, or nucleotides exonucleolytically removed from target nucleic acids, pass through a nanopore. As the target nucleic acids or nucleotides pass through the nanopore, each type of base can be identified by measuring fluctuations in the electrical conductance of the pore, such as described in U.S. Pat. No. 7,001,792; Soni & Meller, Clin. Chem. 53, 1996-2001 (2007); Healy, Nanomed. 2, 459-481 (2007); and Cockroft, et al. J. Am. Chem. Soc. 130, 818-820 (2008), the disclosures of which are incorporated herein by reference in their entireties. Yet other embodiments include detection of a proton released upon incorporation of a nucleotide into an extension product. For example, sequencing based on detection of released protons can use an electrical detector and associated techniques that are commercially available from Ion Torrent (Guilford, Conn., a Life Technologies subsidiary) or sequencing methods and systems described in U.S. Patent Publication Numbers US 2009/0026082 A1, US 2009/0127589 A1, US 2010/0137143 A1, or US 2010/0282617 A1, each of which is incorporated herein by reference in its entirety. Particular embodiments can utilize methods involving the real-time monitoring of DNA polymerase activity. Nucleotide incorporations can be detected through fluorescence resonance energy transfer (FRET) interactions between a fluorophore-bearing polymerase and y-phosphate-labeled nucleotides, or with zeromode waveguides as described, for example, in Levene et al. Science 299, 682-686 (2003); Lundquist et al. Opt. Lett. 33, 1026-1028 (2008); and Korlach et al. Proc. Natl. Acad. Sci. USA 105, 1176-1181 (2008), the disclosures of which are incorporated herein by reference in their entireties. Other suitable alternative techniques include, for example, fluorescent in situ sequencing (FISSEQ), and Massively Parallel Signature Sequencing (MPSS). In particular embodiments, one of the sequencers 100 may be a HiSeq, MiSeq, or HiScanSQ from Illumina (San Diego, Calif.).

In some embodiments, the biological samples may be loaded into the sequencers 100 as sample slides and may be imaged to generate sequence data. For example, reagents that interact with the biological sample fluorescently at particular wavelengths in response to an excitation beam generated by an imaging module and thereby return radiation for imaging. For instance, the fluorescent components may be generated by fluorescently tagged nucleic acids that hybridize to complementary molecules of the components or to fluorescently tagged nucleotides that are incorporated into oligonucleotides in the biological samples using a polymerase. The wavelength at which the dyes of the sample are excited and the wavelength at which they fluoresce may depend upon the absorption and emission spectra of the specific dyes. Such returned radiation may propagate back through directing optics of the imaging module. The imaging module detection optics may be based upon any suitable technology, and may be, for example, a charged coupled device (CCD) sensor that generates pixilated image data based upon photons impacting locations in the device. Alternatively, the imaging module detection optics may be based upon a detector array configured for time delay integration (TDI) operation, a complementary metal oxide semiconductor (CMOS) detector, an avalanche photodiode (APD) detector, a Geiger-mode photon counter, or any other suitable detector. TDI mode detection can be coupled with line scanning as described in U.S. Pat. No. 7,329,860, which is incorporated herein by reference.

### Computing Systems

In some embodiments, the SARJ generator (SARJeant) 300 may involve approach for shifting or distributing certain sequence data analysis features and sequence data storage to a cloud computing environment or cloud-based network. User interaction with sequencing data, genome data, or other types of biological data may be mediated via a central hub that stores and controls access to various interactions with the data. In some embodiments, the cloud computing environment may also provide sharing of protocols, analysis methods, libraries, sequence data as well as distributed processing for sequencing, analysis, and reporting. In some embodiments, the cloud computing environment facilitates modification or annotation of sequence data by users. In some embodiments, the SARJ generator 300 may be implemented in a computer browser, on-demand or on-line.

In some embodiments, software written to perform the SARJ generator 300 as described herein is stored in some form of computer readable medium, such as memory, CD-ROM, DVD-ROM, memory stick, flash drive, hard drive, SSD hard drive, server, mainframe storage system and the like.

In some embodiments, the SARJ generator 300 may be written in any of various suitable programming languages, for example compiled languages such as C, C#, C++, Fortran, and Java. Other programming languages could be script languages, such as Perl, MatLab, SAS, SPSS, Python, Ruby, Pascal, Delphi, R and PHP. In some embodiments, the SARJ generator 300 are written in C, C#, C++, Fortran, Java, Perl, R, Java or Python. In some embodiments, the SARJ generator 300 may be an independent application with data input and data display modules. Alternatively, the SARJ generator 300 may be a computer software product and may include classes wherein distributed objects comprise applications including computational methods as described herein. Further, computer software products may be part of a component software product, including, but not limited to, computer implemented software products associated with sequencing systems offered by Illumina, Inc. (San Diego, Calif.), Applied Biosystems and Ion Torrent (Life Technologies; Carlsbad, Calif.), Roche 454 Life Sciences (Branford, Conn.), Roche NimbleGen (Madison, Wis.), Cracker Bio (Chulung, Hsinchu, Taiwan), Complete Genomics (Mountain View, Calif.), GE Global Research (Niskayuna, N.Y.), Halcyon Molecular (Redwood City, Calif.), Helicos Biosciences (Cambridge, Mass.), Intelligent Bio-Systems (Waltham. Mass.), NAB sys (Providence, R.I.), Oxford Nanopore (Oxford, UK), Pacific Biosciences (Menlo Park, Calif.), and other sequencing software related products for determining sequence from a nucleic acid sample.

In some embodiments, the SARJ generator 300 may be incorporated into pre-existing data analysis software, such as that found on sequencing instruments. An example of such software is the CASAVA Software program (Illumina, Inc., see CASAVA Software User Guide as an example of the program capacity, incorporated herein by reference in its entirety). Software comprising computer implemented methods as described herein are installed either onto a computer system directly, or are indirectly held on a computer readable medium and loaded as needed onto a computer system. Further, the SARJ generator 300 may be located on computers that are remote to where the data is being produced, such as software found on servers and the like that are maintained in another location relative to where the data is being produced, such as that provided by a third party service provider.

An assay instrument, desktop computer, laptop computer, or server which may contain a processor in operational communication with accessible memory comprising instructions for implementation of the SARJ generator 300. In some embodiments, a desktop computer or a laptop computer is in operational communication with one or more computer readable storage media or devices and/or outputting devices. An assay instrument, desktop computer and a laptop computer may operate under a number of different computer based operational languages, such as those utilized by Apple based computer systems or PC based computer systems. An assay instrument, desktop and/or laptop computers and/or server system may further provide a computer interface for creating or modifying experimental definitions and/or conditions, viewing data results and monitoring experimental progress. In some embodiments, an outputting device may be a graphic user interface such as a computer monitor or a computer screen, a printer, a hand-held device such as a personal digital assistant (i.e., PDA, Blackberry, iPhone), a tablet computer (e.g., iPAD®), a hard drive, a server, a memory stick, a flash drive and the like.

A computer readable storage device or medium may be any device such as a server, a mainframe, a super computer, a magnetic tape system and the like. In some embodiments, a storage device may be located onsite in a location proximate to the assay instrument, for example adjacent to or in close proximity to, an assay instrument. For example, a storage device may be located in the same room, in the same building, in an adjacent building, on the same floor in a building, on different floors in a building, etc. in relation to the assay instrument. In some embodiments, a storage device may be located off-site, or distal, to the assay instrument. For example, a storage device may be located in a different part of a city, in a different city, in a different state, in a different country, etc. relative to the assay instrument. In embodiments where a storage device is located distal to the assay instrument, communication between the assay instrument and one or more of a desktop, laptop, or server is typically via Internet connection, either wireless or by a network cable through an access point. In some embodiments, a storage device may be maintained and managed by the individual or entity directly associated with an assay instrument, whereas in other embodiments a storage device may be maintained and managed by a third party, typically at a distal location to the individual or entity associated with an assay instrument. In embodiments as described herein, an outputting device may be any device for visualizing data.

An assay instrument, desktop, laptop and/or server system may be used itself to store and/or retrieve computer implemented software programs incorporating computer code for performing and implementing computational methods as described herein, data for use in the implementation of the computational methods, and the like. One or more of an assay instrument, desktop, laptop and/or server may comprise one or more computer readable storage media for storing and/or retrieving software programs incorporating computer code for performing and implementing computational methods as described herein, data for use in the implementation of the computational methods, and the like. Computer readable storage media may include, but is not limited to, one or more of a hard drive, a SSD hard drive, a CD-ROM drive, a DVD-ROM drive, a floppy disk, a tape, a flash memory stick or card, and the like. Further, a network including the Internet may be the computer readable storage media. In some embodiments, computer readable storage media refers to computational resource storage accessible by a computer network via the Internet or a company network offered by a service provider rather than, for example, from a local desktop or laptop computer at a distal location to the assay instrument.

In some embodiments, computer readable storage media for storing and/or retrieving computer implemented software programs incorporating computer code for performing and implementing computational methods as described herein, data for use in the implementation of the computational methods, and the like, is operated and maintained by a service provider in operational communication with an assay instrument, desktop, laptop and/or server system via an Internet connection or network connection.

In some embodiments, a hardware platform for providing a computational environment comprises a processor (i.e., CPU) wherein processor time and memory layout such as random access memory (i.e., RAM) are systems considerations. For example, smaller computer systems offer inexpensive, fast processors and large memory and storage capabilities. In some embodiments, graphics processing units (GPUs) can be used. In some embodiments, hardware platforms for performing computational methods as described herein comprise one or more computer systems with one or more processors. In some embodiments, smaller computer are clustered together to yield a supercomputer network.

In some embodiments, computational methods as described herein are carried out on a collection of inter- or intra-connected computer systems (i.e., grid technology) which may run a variety of operating systems in a coordinated manner. For example, the CONDOR framework (University of Wisconsin-Madison) and systems available through United Devices are exemplary of the coordination of multiple stand-alone computer systems for the purpose dealing with large amounts of data. These systems may offer Perl interfaces to submit, monitor and manage large sequence analysis jobs on a cluster in serial or parallel configurations.

### Definitions

As used herein, the singular forms “a”, “and”, and “the” include plural referents unless the context clearly dictates otherwise. Thus, for example, reference to “a sequence” may include a plurality of such sequences, and so forth. All technical and scientific terms used herein have the same meaning as commonly understood to one of ordinary skill in the art to which this invention belongs unless clearly indicated otherwise.

As used herein, the term “data strings” refers to a group or list of characters derived from a data set. As used herein, the term “collection,” when used in reference to “data strings” refers to one or more data strings. A collection can comprise one or more data strings, each data string comprising characters derived from a data set A collection of data strings can be made up of a group or list of characters from more than one data set, such that a collection of data strings can be, for example, a collection of data strings from two or more different data sets. Or, a collection of data strings can be derived from one data set. As such, a “collection of characters” is one or more letters, symbols, words, phrases, sentences, or data related identifiers collated together, wherein said collation creates a data string or a string of characters. Further, a “plurality of data strings” refers to two or more data strings. In one example, a data string can form a row of characters and two or more rows of characters can be aligned to form multiple columns. For example, a collection of 10 strings, each string having 20 characters, can be aligned to form 10 rows and 20 columns.

As used herein, a “subsequence”, “substring”, “prefix” or “suffix” of a string represents a subset of characters, letters, words, etc, of a longer list of characters, letters, words, etc., (i.e., the longer list being the sequence or string) wherein the order of the elements is preserved. A “prefix” typically refers to a subset of characters, letters, numbers, etc. found at the beginning of a sequence or string, whereas a “suffix” typically refers to a subset of characters, letters, numbers, etc. found at the end of a string. Substrings are also known as subwords or factors of a sequence or string.

As used herein, the term “protocol” refers to a method, step or instruction or set of methods, steps or instructions performed in completing a task, such as preparing a biological sample. A sample preparation protocol typically includes, for example, a step-by-step set of instructions to complete a task. The protocol may contain only a sub-set of the steps needed to complete the task. The set of instructions can be performed entirely in a manual manner, entirely in an automated manner, or a mixture of one or more manual and automated steps may be performed in combination. For example, a sample preparation protocol may have as an initial step the manual introduction of a nucleic acid sample or cell lysate into an inlet port of a sample preparation cartridge, after which the rest of the protocol is performed in an automated manner by a device.

As used herein, the term “sample preparation related data” refers to information related to a sample preparation procedure, including executable instructions for carrying out a sample preparation procedure on a device, and/or data related to a specific sample preparation procedure such as sample identification, date, time and other particular details of sample preparation procedure. For example, sample preparation related data can include sample preparation recipe/protocol identification, sample preparation cartridge identification, cartridge preparation identification, sample preparation instrument identification, and other parameters. In some embodiments, sample preparation related data is input or provided by a user to a sample preparation device. In some embodiments, sample preparation related data is provided by a user to a third party, or to a cloud computing environment. In some embodiments, sample preparation related data is provided from a cloud computing environment or a third party to a sample preparation device.

As used herein, the term “sequencing related data” refers to information provided in connection with sequencing. For example, sequencing related data can include, but is not limited to, flowcell identification, sequencing cartridge identification, sequencing instrument identification, and sequencing parameters. Sequencing related data can be provided, for example, by a user, a third party, or by a sequencing instrument. In some embodiments, sequencing related data is input or provided by a user to a sample preparation device. In some embodiments, sequencing related data is provided by a user to a third party, or to a cloud computing environment. In some embodiments, sequencing related data is provided from a cloud computing environment or a third party to a sample preparation device.

As used herein, the term “sample manifest” refers to a list including one or more of the samples being processed in a sample preparation procedure. The sample manifest may include, for example, identifier numbers or other identifying information for the one or more samples. In some embodiments, the samples on the sample manifest are processed in parallel. In some embodiments, the samples on the sample manifest are processed consecutively.

As used herein, the term “user” may refer to the owner of the sequence data, a researcher or clinician who uploads the sequence data to the cloud, or an original researcher who performed the sequencing run, a doctor or clinician who is handling a particular aspect of a patient's care, a primary care physician, oncologist and genetic counselor who are caring for the individual whose sequence is being accessed. Different users can have different permission levels with regard to the number and types of annotations and modifications they can make to the files.

### EXAMPLES

The following examples are offered to illustrate but not to limit the invention. In order to facilitate understanding, the specific embodiments are provided to help interpret the technical proposal, that is, these embodiments are only for illustrative purposes, but not in any way to limit the scope of the invention. Unless otherwise specified, embodiments do not indicate the specific conditions, are in accordance with the conventional conditions or the manufacturer's recommended conditions.

### Example 1

The output file, Sample Analysis Results JSON (SARJ) file, was generated as a standard text based JavaScript Object Notation (JSON) file. The content of the SARJ file included:

1. Checksum—checksum of the data section, can be salted to safeguard from undesired user modifications to the file.

2. Data Section
a. Schema version.
b. Sample information—a set of properties for describing the sample, including disease information.
c. Software configuration information—set of properties capturing version information for upstream software such as the analysis pipeline.
d. Quality control information
   i. Run metrics.
   ii. Sequencing library status (e.g. RNA and DNA libraries).
   iii. QC metrics.

3. Variants—lists of data for multiple variant types, where the type of variants included depends on the analysis pipeline (e.g. small variants, copy number variation (CNV), fusions, splice variants).

4. Biomarkers—sets of properties grouped by biomarker type (e.g. tumor mutational burden, micro satellite instability).

While certain embodiments of the inventions have been described, these embodiments have been presented by way of example only, and are not intended to limit the scope of the disclosure. Indeed, the novel methods and systems described herein may be embodied in a variety of other forms. Furthermore, various omissions, substitutions and changes in the systems and methods described herein may be made without departing from the spirit of the disclosure. The accompanying claims and their equivalents are intended to cover such forms or modifications as would fall within the scope and spirit of the disclosure. Accordingly, the scope of the present inventions is defined only by reference to the appended claims.

Features, materials, characteristics, or groups described in conjunction with a particular aspect, embodiment, or example are to be understood to be applicable to any other aspect, embodiment or example described in this section or elsewhere in this specification unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The protection is not restricted to the details of any foregoing embodiments. The protection extends to any novel one, or any novel combination, of the features disclosed in this specification (including any accompanying claims, abstract and drawings), or to any novel one, or any novel combination, of the steps of any method or process so disclosed.

Furthermore, certain features that are described in this disclosure in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations, one or more features from a claimed combination can, in some cases, be excised from the combination, and the combination may be claimed as a subcombination or variation of a subcombination.

Moreover, while operations may be depicted in the drawings or described in the specification in a particular order, such operations need not be performed in the particular order shown or in sequential order, or that all operations be performed, to achieve desirable results. Other operations that are not depicted or described can be incorporated in the example methods and processes. For example, one or more additional operations can be performed before, after, simultaneously, or between any of the described operations. Further, the operations may be rearranged or reordered in other implementations. Those skilled in the art will appreciate that in some embodiments, the actual steps taken in the processes illustrated and/or disclosed may differ from those shown in the figures. Depending on the embodiment, certain of the steps described above may be removed, others may be added. Furthermore, the features and attributes of the specific embodiments disclosed above may be combined in different ways to form additional embodiments, all of which fall within the scope of the present disclosure. Also, the separation of various system components in the implementations described above should not be understood as requiring such separation in all implementations, and it should be understood that the described components and systems can generally be integrated together in a single product or packaged into multiple products. For example, any of the components for an energy storage system described herein can be provided separately, or integrated together (e.g., packaged together, or attached together) to form an energy storage system.

For purposes of this disclosure, certain aspects, advantages, and novel features are described herein. Not necessarily all such advantages may be achieved in accordance with any particular embodiment. Thus, for example, those skilled in the art will recognize that the disclosure may be embodied or carried out in a manner that achieves one advantage or a group of advantages as taught herein without necessarily achieving other advantages as may be taught or suggested herein.

Conditional language, such as “can,” “could,” “might,” or “may,” unless specifically stated otherwise, or otherwise understood within the context as used, is generally intended to convey that certain embodiments include, while other embodiments do not include, certain features, elements, and/or steps. Thus, such conditional language is not generally intended to imply that features, elements, and/or steps are in any way required for one or more embodiments or that one or more embodiments necessarily include logic for deciding, with or without user input or prompting, whether these features, elements, and/or steps are included or are to be performed in any particular embodiment.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require the presence of at least one of X, at least one of Y, and at least one of Z.

Language of degree used herein, such as the terms “approximately,” “about,” “generally,” and “substantially” represent a value, amount, or characteristic close to the stated value, amount, or characteristic that still performs a desired function or achieves a desired result.

The scope of the present disclosure is not intended to be limited by the specific disclosures of preferred embodiments in this section or elsewhere in this specification, and may be defined by claims as presented in this section or elsewhere in this specification or as presented in the future. The language of the claims is to be interpreted broadly based on the language employed in the claims and not limited to the examples described in the present specification or during the prosecution of the application, which examples are to be construed as non-exclusive.

What is claimed is:

1. A computer-implemented method of generating a custom file, comprising:
receiving a query for information associated with a desired sample;
determining a schema for structuring the custom file;
obtaining, according to the schema, a plurality of nucleic acid sequencing analysis files, wherein each one of the plurality of nucleic acid sequencing analysis files comprises nucleic acid sequence information, genetic variant information, gene expression information, or any combination thereof, of a plurality of biological samples, wherein the plurality of biological samples comprise the desired sample;
for each one of the plurality of nucleic acid sequencing analysis files:
determining, according to the schema, a plurality of data objects in the nucleic acid sequencing analysis file to be stored in the custom file;
determining, according to the schema, a plurality of custom data fields in the custom file to store the data objects; and
storing the data objects in the custom data fields;
generating a checksum by evaluating a cryptographic hash function for a portion of the custom file according to the schema; and
storing the checksum in the custom file.

2. The method of claim 1, wherein determining a schema for structuring the custom file comprises:
choosing a schema from a plurality of pre-defined schemas;
optionally, receiving user modifications for modifying the schema; and
storing the user modifications and a version value associated with the schema in the custom file.

3. The method of claim 1, wherein obtaining, according to the schema, a plurality of nucleic acid sequencing analysis files comprises:
searching a database for a plurality of files comprising one or more keywords specified by the schema; and
copying the plurality of files.

4. The method of claim 1, wherein determining, according to the schema, a plurality of data objects in the nucleic acid sequencing analysis file to be stored in the custom file comprises:
parsing the nucleic acid sequencing analysis file;
identifying, according to the schema, the plurality of data objects to be stored; and
extracting the plurality of data objects.

5. The method of claim 1, wherein each of the nucleic acid sequencing analysis files further comprises at least one of: sequencing device condition, sequencing related data, analysis software information, analysis pipeline information, base calls, run quality control metrics, DNA quality control metrics, RNA quality control metrics, DNA small variants outputs, copy number variant outputs, RNA fusion outputs, DNA fusion outputs, splice variant outputs, tumor mutational burden biomarker outputs, and microsatellite instability biomarker outputs.

6. The method of claim 5, wherein the sequencing device condition comprises sequencing parameters and/or information about errors in the sequencing device.

7. The method of claim 1, wherein each of the nucleic acid sequencing analysis files further comprises at least one of: sample preparation related data, sample identification number, sample manifest, patient identify, tissue type, genomic area of interest, disease information, and treatment information.

8. The method of claim 1, further comprising:
receiving a user input associated with the desired sample;
determining, according to the schema, a plurality of data objects in the user input to be stored in the custom file;
determining, according to the schema, a plurality of custom data fields in the custom file to store the data objects; and
storing the data objects in the custom data fields.

9. The method of claim 8, wherein the user input associated with the desired sample comprises at least one of: sample preparation related data, sample identification number, sample manifest, patient identify, tissue type, genomic area of interest, disease information, and treatment information.

10. The method of claim 1, wherein the cryptographic hash function is a MD5 hash function, a MD6 hash function, a SHA-1 hash function, a SHA-256 hash function, or a SHA-512 hash function.

11. The method of claim 1, further comprising:
generating a verification value by adding or multiplying the checksum by a number; and
storing the verification value in the custom file.

12. The method of claim 11, wherein the number is π.

13. The method of claim 1, wherein the portion of the custom file according to the schema comprises a plurality of custom data fields declared by the schema as not permitting user corrections.

14. The method of claim 13, further comprising:
generating an additional checksum by evaluating a cryptographic hash function for an additional portion of the custom file according to the schema, wherein the additional portion of the custom file comprises a plurality of custom data fields declared by the schema as permitting user corrections; and
storing the additional checksum in the custom file.

15. The method of claim 1, further comprising:
receiving and storing a plurality of user changes to a plurality of custom data fields;
updating the checksum by re-evaluating the cryptographic hash function for the portion of the custom file according to the schema; and
storing the updated checksum in the custom file.

16. The method of claim 1, wherein some of the nucleic acid sequencing analysis files are compressed.

17. The method of claim 1, further comprising compressing and/or encrypting the custom file.

18. The method of claim 1, wherein the custom file is in text-based JavaScript Object Notation (JSON) format or binary JSON format.

19. The method of claim 1, wherein each of the nucleic acid sequencing analysis files is in one of JSON, CSV, TSV, XML, NirvanaJSON, VCF, CSVVCF, or SpliceJSON format.

20. The method of claim 1, wherein the method is implemented in a cloud computing environment.

21. A database comprising a plurality of files, wherein each of the plurality of files is generated according to the method of claim 1.

22. A system for generating a custom file, comprising:
a memory storing instructions to implement the method of claim 1; and
one or more processors configured to execute the instructions.

23. A computer program product for generating a custom file, comprising a computer readable storage medium having program instructions to implement the method of claim 1.
